(19) United States
(12) Patent Application Publication
(10) Pub. No.: US 2006/0212058 A1
(43) Pub. Date: Sep. 21, 2006

(54) Disposable safety surgical blade

(76) Inventors: Ilija Djordjevic, East Granby, CT (US); Sushil K. Kanwar, West Hartford, CT (US)

Correspondence Address: ALIX YALE & RISTAS LLP, 750 MAIN STREET, SUITE 1400, HARTFORD, CT, 06103, US

(21) Appl. No.: 11/374,526

(22) Filed: Mar. 13, 2006

Related U.S. Application Data
(60) Provisional application No. 60/663,375, filed on Mar. 18, 2005.

(51) Int. Cl. A61B 17/32

(52) U.S. Cl. 606/167

(57) ABSTRACT

A surgical scalpel assembly includes a blade carrier, a blade, a protective sleeve and a handle. The blade carrier has a number of notches and the protective sleeve has a depending tooth. The user positions the sleeve relative to the blade carrier and/or the handle by positioning the tooth to engage the notches. The blade carrier, blade, and protective sleeve are pre-assembled as a cartridge. A high volume yet safe method of assembly is also described.

### RELATED APPLICATION

This application claims priority under 35 U.S.C. § 119(e) based on provisional application No. 60/663,375 filed Mar. 18, 2005.

### BACKGROUND OF THE INVENTION

The present invention pertains to surgical scalpels, and in particular, such scalpels of the type that have a protective shield or the like whereby the blade can be selectively exposed or covered in use, then disposed of.

Many scalpels of this type are known, such as described in the following U.S. patents and U.S. published applications:

| | | |
|---|---|---|
| 3,793,726 | 5,330,494 | 5,527,329 |
| 5,868,771 | 5,919,201 | 5,938,676 |
| 5,938,675 | 941,892 | 6,053,929 |
| 6,626,925 | 2004/0243161 | D490,153 |

The general concept of a blade carrier attachable to a handle and to the blade itself, with an integral or separate protective sleeve or guide for the blade that is moveable to at least two positions, is well known. Moreover, the further refinement of the blade carrier with associated blade being detachable from the handle for disposal, while recovering the handle for sterilization and reuse is also known. U.S. Pat. No. 5,919,201 embodies this refinement in a combination of a handle, a cartridge removably mounted to the handle, a blade attached to a blade holder in the cartridge and a shield mounted on the blade holder.

The known devices may all perform their intended function, but none exhibits an ideal combination of low cost components and manufacturability, with simplicity yet reliability of use.

### SUMMARY OF THE INVENTION

A significantly improved combination of such desirable attributes is achieved with the present invention. In general aspect, the invention comprises a blade carrier having a lower edge defining a plurality of notches, a handle having a notch on the lower edge, and a protective sleeve having a depending tooth for engaging the notches on the carrier and the handle. The user slides the sleeve and positions the tooth for selectively establishing and reestablishing at least three detent conditions of (1) pre-assembly, (2) assembly to complete attachment of the handle to the carrier, and (3) a cutting condition.

Another aspect of the invention is directed to a high volume yet safe assembly technique for the carrier, blade, and sleeve as a cartridge or the like for later attachment to the handle.

Those familiar with this field of endeavor will appreciate that one or more of advantages of the preferred embodiment include: (1) minimized waste by minimizing of the amount of material to be disposed; (2) lower tooling cost by locating of all the blade and carrier retention features on the same side as well as minimizing the number of tolerance sensitive features; (3) independent optimization of the shape and weight of the reusable handle; (4) prevention of accidental reuse of contaminated blade; and (5) easy reclaiming of material used for the blade as well as blade carrier and protection sleeve.

### BRIEF DESCRIPTION OF THE DRAWINGS

The preferred embodiments will be described with reference to the accompanying drawings, in which:

FIG. 1 is an exploded view of the three main components of blade, blade carrier, and protective sleeve that are ultimately assembled with a handle to form the scalpel;

FIG. 2 is an elevation view of the three components shown in FIG. 1, pre-assembled as a cartridge before attachment to a handle;

FIG. 3 is an enlarged view of the blade carrier component, showing one suitable pattern of three notches on the bottom edge;

FIG. 4 is an exploded view of the handle and the pre-assembly cartridge of FIG. 2, in position for attachment together;

FIG. 5 is an elevation view, partly in section, of the assembled scalpel in a storage condition;

FIG. 6 is view similar to FIG. 5, showing the sleeve retracted to expose the blade for use during surgery;

FIG. 7 is a view similar to FIG. 6, showing the sleeve displaced fully forward, to cover the blade permanently and permit detachment of the cartridge from the handle for disposal;

FIG. 8 is an elevation view corresponding to FIG. 2, of an alternative embodiment wherein the blade has a different shape, the blade carrier has a different notch pattern, and the sleeve has a different support for the tooth;

FIG. 9 is a view similar to FIG. 5, partially in longitudinal section, showing the scalpel with the pre-assembly cartridge of FIG. 8 in the storage condition;

FIG. 10 is a perspective view of an alternative embodiment of the sleeve of FIG. 1 wherein the tooth has a different shape and is located at a different position on the sleeve;

FIG. 11a is a longitudinal section view of the embodiment of FIG. 10;

FIG. 11b is a cross-sectional view of the proximal end of the embodiment of FIG. 10;

FIG. 12 is a longitudinal view, partially in section, of FIG. 9 where the sleeve is fully forward in the disposal condition;

FIG. 13 is a side view of an alternative embodiment of the elongated blade assembly wherein the means for attaching the blade to the blade carrier is different; and

FIGS. 14A-14E illustrate the preferred method for assembling the scalpel of the embodiment shown in FIGS. 8 and 9.

### DETAILED DESCRIPTION

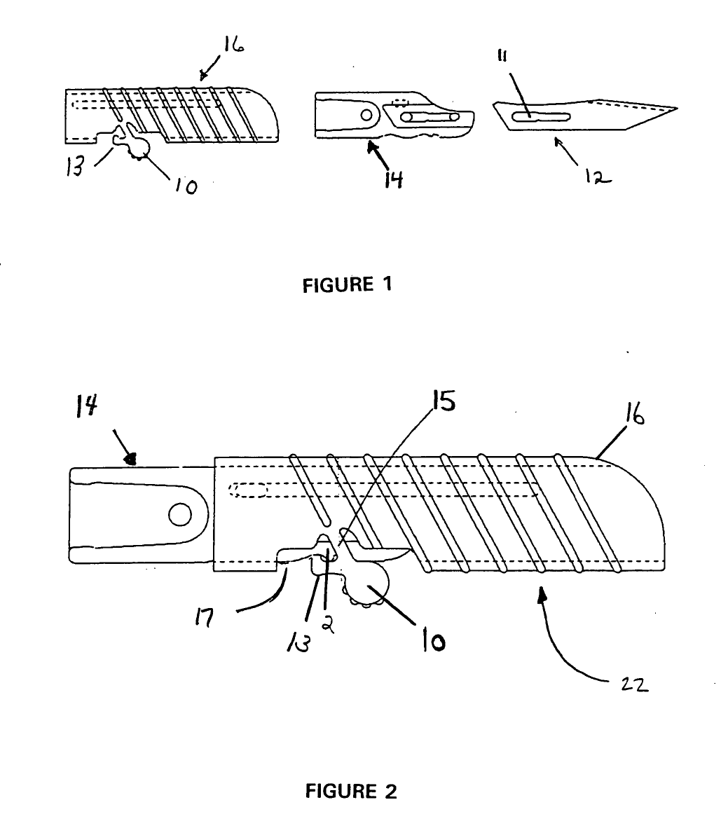

The present invention is directed to a safety scalpel assembly to be mounted on a customized reusable handle, preferably of stainless steel. As illustrated in FIG. 1, the pre-assembly or cartridge of the safety scalpel comprises three components: sleeve 16, blade carrier 14, and blade 12. FIG. 2 illustrates the sleeve 16, the blade carrier 14 and blade 12 (hidden from view) pre-assembled as a cartridge 22. The blade 12 may be any number of different shapes and sizes.

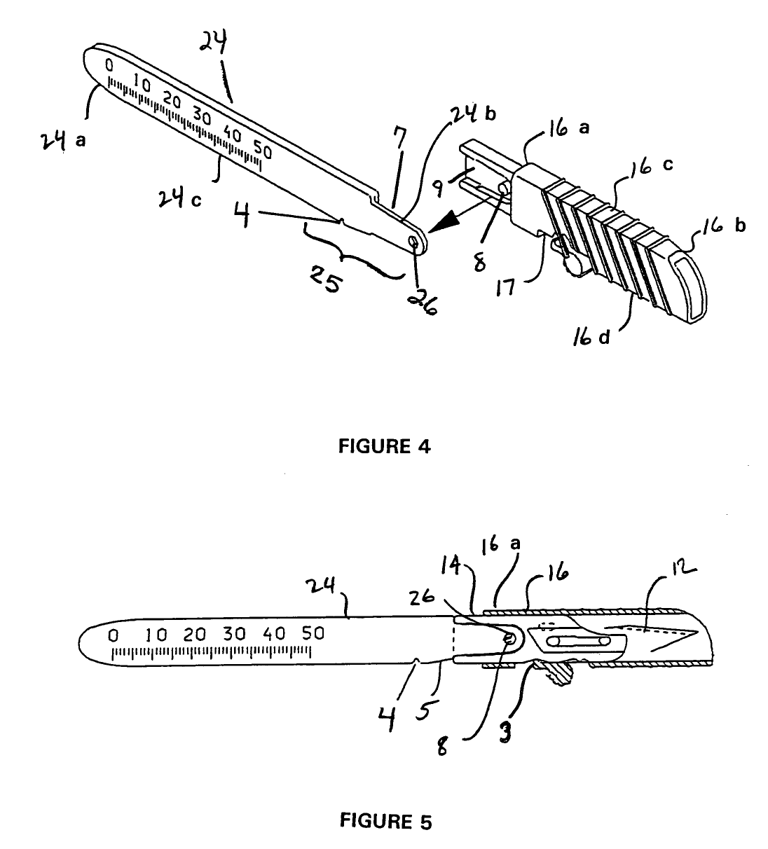

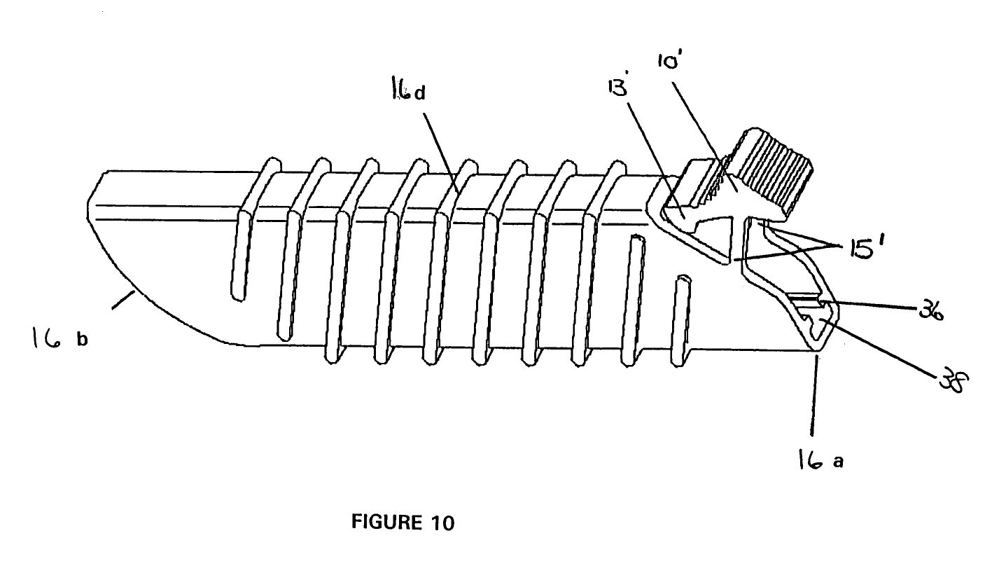

It is an aspect of the invention that blade carrier 14 and sleeve 16 are manufactured from durable materials able to withstand repeated sterilization by, typically, the fabricator. In one preferred embodiment the blade carrier 14 is injection molded using a suitable thermoplastic material. It is also preferred that sleeve 16 is transparent thermoplastic material to allow post-assembly visual inspection and identification of the blade 12. In another embodiment sleeve 16 can be non-transparent. As best seen in FIGS. 4 and 10, sleeve 16 is substantially tubular with two open ends (a proximal end 16a and a distal end 16b), and a closed top 16c and bottom 16d except for a bottom opening 17 intermediate the ends.

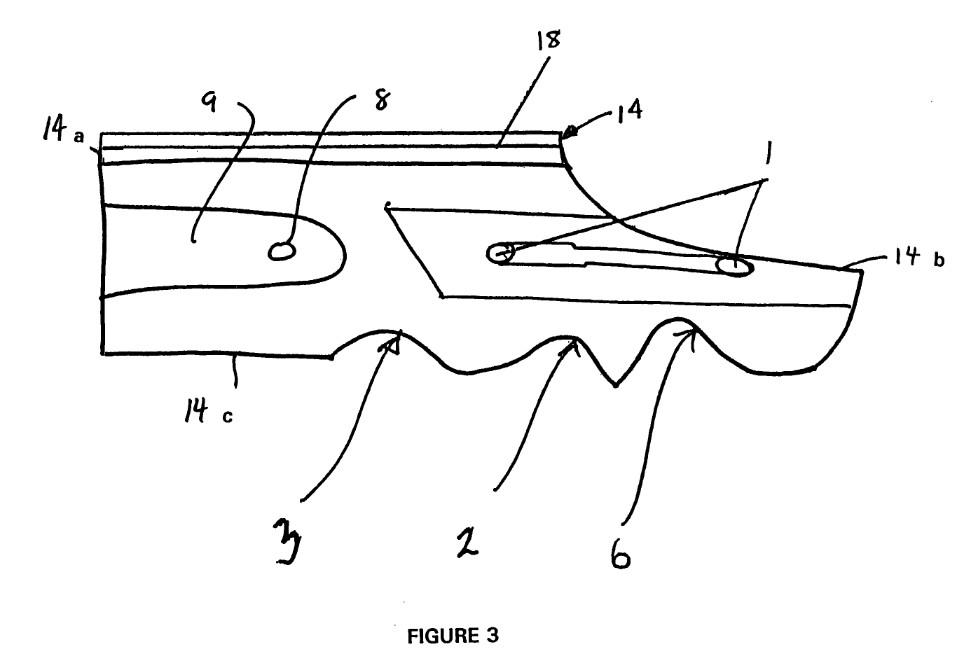

In an exemplary embodiment illustrated in FIG. 3 the blade carrier 14 has a proximal portion 14a, a distal portion 14b, and a lower edge 14c having three representative notches 3, 2, and 6, the functions of which will be described below. A pair of longitudinally spaced blade guide pins 1 are located on the distal portion of blade carrier 14, and project laterally. Handle pin 8 projects from the forward portion of mounting pocket or recess 9, which is located on the proximal portion of blade carrier 14. Blade carrier 14 is further provided with a guide channel 18 which is configured to interfit with a guide track of sleeve 16. The guide channel 18 allows the sleeve 16 to slide along the blade carrier 14 in a controlled and smooth manner.

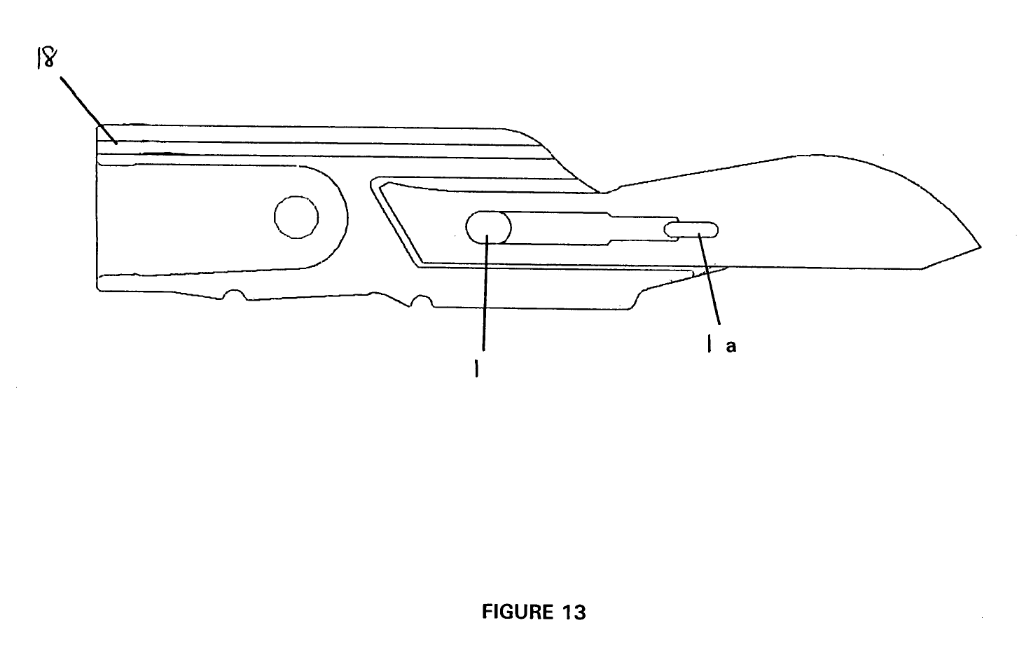

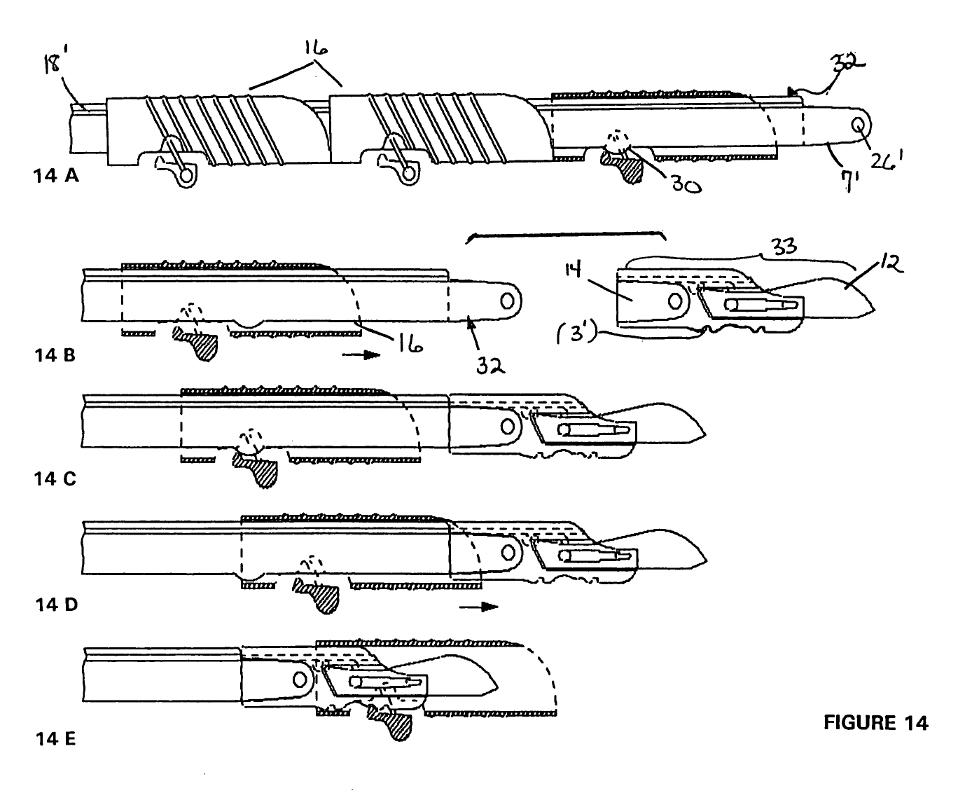

The distal portion of blade carrier 14 is fastened to the blade 12 by inserting guide pins 1 into an opening 11 on the blade 12, which are then riveted to securely attach the blade 12 to the blade carrier 14 to form the elongated blade assembly 33 (an example of which is seen in FIG. 14B). Blade 12 may be fastened to blade carrier 14 using thermal or ultrasound riveting methods. The attached blade is thus offset relative to the blade carrier centerline.

FIG. 13 illustrates another embodiment of the blade carrier which has a guide pin 1 longitudinally spaced from a second guide pin 1a that projects laterally. For example, guide pin 1a may be configured as a laterally projecting hook. It is an aspect of this embodiment that in fastening the blade to the blade carrier, only guide pin 1 is riveted during the fastening process.

FIGS. 2, 8, and 10 illustrate various embodiments of the sleeve 16. FIG. 2 shows an embodiment of sleeve 16 having a cantilevered retention tooth 13 with an associated release button 10. Preferably, the cantilevered retention tooth 13 and release button 10 is connected to sleeve 16 by two leg portions 15 integral with the body of the sleeve and extending downwardly and obliquely forward alongside the opening 17 to form an integral shank. Legs 15 are configured to urge the retention tooth 13 into resilient contact with the bottom edge 14c of the blade carrier 14, strongly enough for the tooth 13 to enter and exert pressure against any of the notches that are encountered as the sleeve 16 slides relative to the blade assembly. The release button or tab 10 is also integrally formed on the legs 15, below the tooth and the bottom surface of the sleeve. The geometric relationship between the release button 10 and legs 15 are such that a forward pressing of the button 10 bends the legs 15 forward and thus pulls the tooth 13 downwardly, away from the bottom edge 14c of the blade carrier. It is an aspect of this embodiment that the tooth 13 and the button 10 are at all times below or within the opening 17 in the bottom of the sleeve. It is also preferred that sleeve 16 have an interior with a channel 38 and rail 36 configuration (an example of which is seen in FIG. 10).

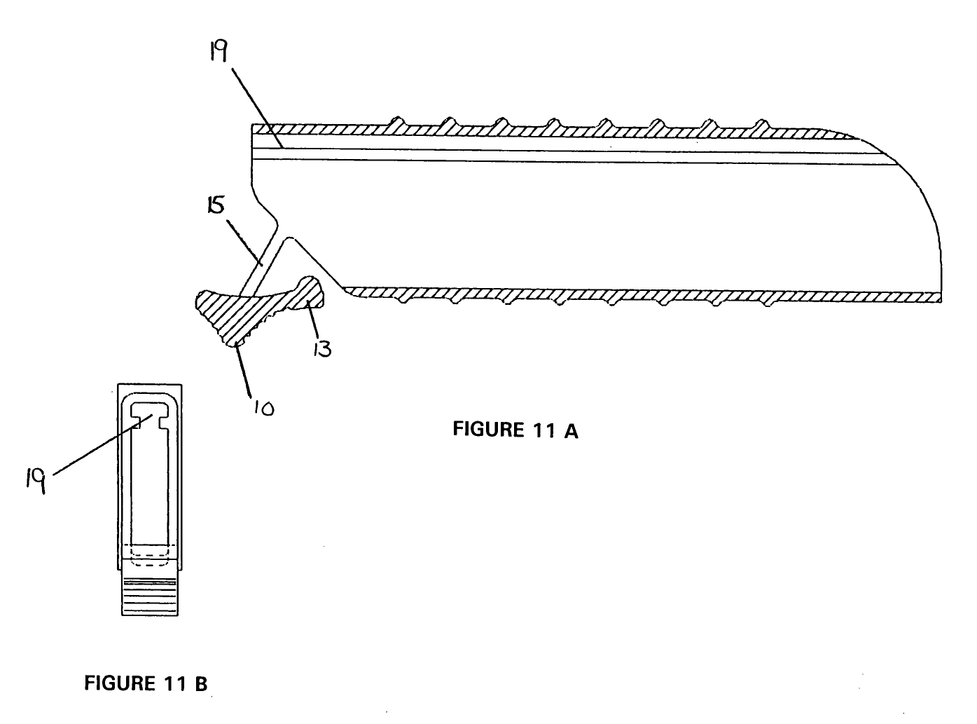

FIG. 10 illustrates another embodiment of the sleeve 16 having two legs 15′, a cantilevered tooth 13′, and an associated release button 10′ positioned at the opening at the proximal end 16a. As seen in FIG. 10, according to this embodiment there is no opening 17 along the bottom 16d of the sleeve. Retention tooth 13′ and release button 10′ function and are manipulated in a manner analogous to the embodiment of FIG. 2. FIG. 10 also illustrates the channel 38 and rail 36 which together form a guide track 19 that is complementary to the guide channels 18 on the blade carrier 14. Guide track 19 is also illustrated in FIGS. 11a and 11b. The channel and rail configuration of guide track 19 allows for sleeve 16 to be moved relative to the blade carrier 14 in a controlled and stable manner.

The pre-assembly cartridge 22 is complete after the distal end of sleeve 16 is pushed over the proximal portion of blade carrier 14 until the retention tooth 13 of the sleeve 16 engages with the notch 2 of the blade carrier 14 as seen in FIGS. 2 and 3. Thereafter, the pre-assembly cartridge 22 is sterilized and stored in a sealed container.

As illustrated in FIG. 4, handle 24 has proximal and distal portions 24a, 24b, the latter having a mounting end 25 configured with an off-center mounting tongue 7 and a locating bore 26 suitable to engage the handle pin 8 located on the proximal portion of blade carrier 14.

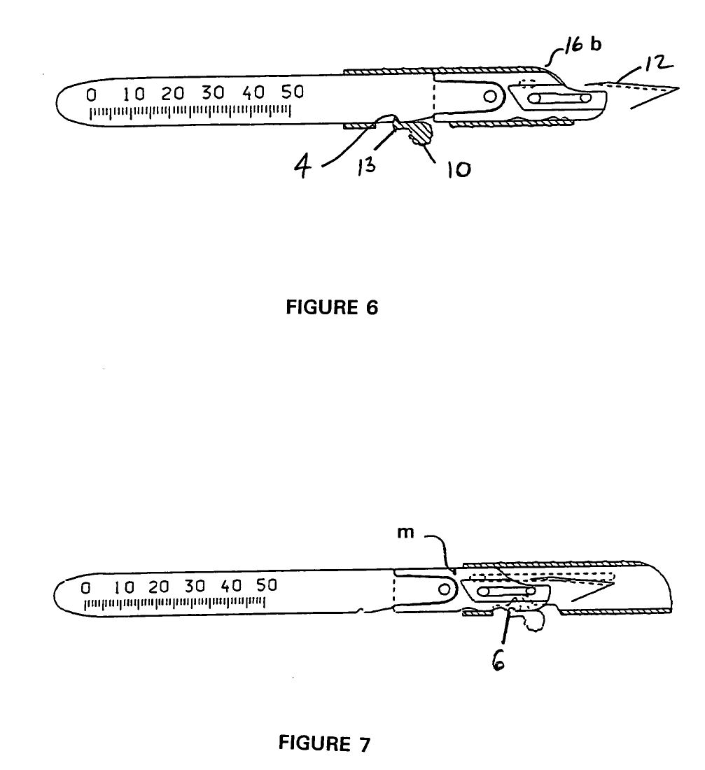

Assembly of the surgical instrument is completed by attaching the distal portion of handle 24 to the pre-assembly cartridge 22 by inserting the handle pin 8 of the blade carrier 14 into the locating bore 26 of the handle 24. As illustrated in FIG. 4, the distal portion of handle 24 and proximal portion of blade carrier 14 are laterally pushed together until the mounting tongue 7 completely engages the mounting pocket 9 of the blade carrier 14. The handle and the blade are thus offset on the same side of the blade carrier. The proximal end of sleeve 16 is then pulled toward the handle 24, until the retention tooth 13 engages with the notch 3 of the blade carrier 14. This configuration is the assembled condition and, as shown in FIG. 6, the blade 12 remains protected by virtue of its location within the sleeve 16. In the assembled condition, the blade carrier 14 is firmly retained on the handle 24 by the overlap of the proximal portion 16a with the distal end 24b of the handle which includes mounting tongue 7, pin 8, and bore 26.

Handle 24 also has a notch 4 on lower edge 24c for engaging the retention tooth 13 of sleeve 16 during periods when the instrument is in use. Specifically, when the retention tooth 13 is engaged with notch 4 the blade 12 is exposed for cutting. This cutting condition is best seen in FIG. 6. The blade 12 is exposed by sliding the proximal end of sleeve 16 in a longitudinal direction toward the handle 24 until the retention tooth 13 engages with notch 4 on the handle 24. As depicted in FIG. 6, in the cutting condition the distal end 16b of the sleeve 16 is positioned behind the blade 12 thus exposing the blade 12 for use. In a preferred embodiment the sloped portion 5 of lower edge 24c of the handle 24 forward of notch 4 facilitates the engagement of retention tooth 13 and notch 4.

During use of the instrument, it is often desirable to temporarily cover and/or withdraw the blade 12. This can be accomplished by depressing the button 10 located adjacent to the retention tooth 13. This action unlocks the sleeve 16 and allows it to slide forward to the point where retention tooth 13 rests in notch 3 of the blade carrier 14. This is the protected condition and in this condition, the blade 12 is temporarily protected by the distal portion of sleeve 14. This temporary protection feature allows for safer person-to-person transfer of the scalpel during a surgical procedure. Moreover, the blade can be repeatedly covered and uncovered as necessary.

At the conclusion of a surgical procedure, the cartridge 22 can be safely and quickly removed while retaining the handle 24. This is achieved by depressing the button 10 and pushing the distal portion of sleeve 16 longitudinally away from handle 24 until an audible click is heard. As shown in FIG. 7, retention tooth 13 of sleeve 16 engages notch 6 on the blade carrier 14 and effectively locks the sleeve 16 in this position. Once the sleeve 16 is in the locked condition it is very difficult to return to the other conditions.

Having sleeve 16 in the locked condition prevents re-exposure of the blade 12, which in turn makes accidental reuse of a contaminated blade virtually impossible. In another embodiment, pushing the distal portion of sleeve 16 so as to engage retention tooth 13 with notch 6 exposes a suitable “previous use” marking M (also seen in FIG. 7) which provides an additional measure of safety. Exposing a “previous use” marking in this manner would allow would-be users to quickly ascertain by visual inspection that a particular surgical instrument should not be used. Once the sleeve 16 is locked, cartridge 22 is then completely disengaged laterally from the handle and disposed of.

It is noteworthy that once the scalpel has been assembled, the sliding of the sleeve between the protected condition and the use condition can be accomplished by manipulation with the fingers of the same hand as is holding the blade. Likewise, at the conclusion of the surgical procedure, the sleeve can be pushed to the disposal position with only the fingers of the hand that holds the handle. This procedure avoids any possibility that the other hand might come into contact with the exposed blade.

If the sleeve 16 and the blade carrier 14 are manufactured from the same transparent material, the type and size of the blade 12 would be indicated by an appropriate marking located on the blade carrier 14 and would be visible through the sleeve 16. This embodiment would simplify recycling and/or disposal of both the blade 12 and the sleeve 16, as no separation by color or material would be required. In another aspect, the blade 12 could be re-sharpened and re-sterilized and the sleeve 16 and carrier 14 could be reused/recycled.

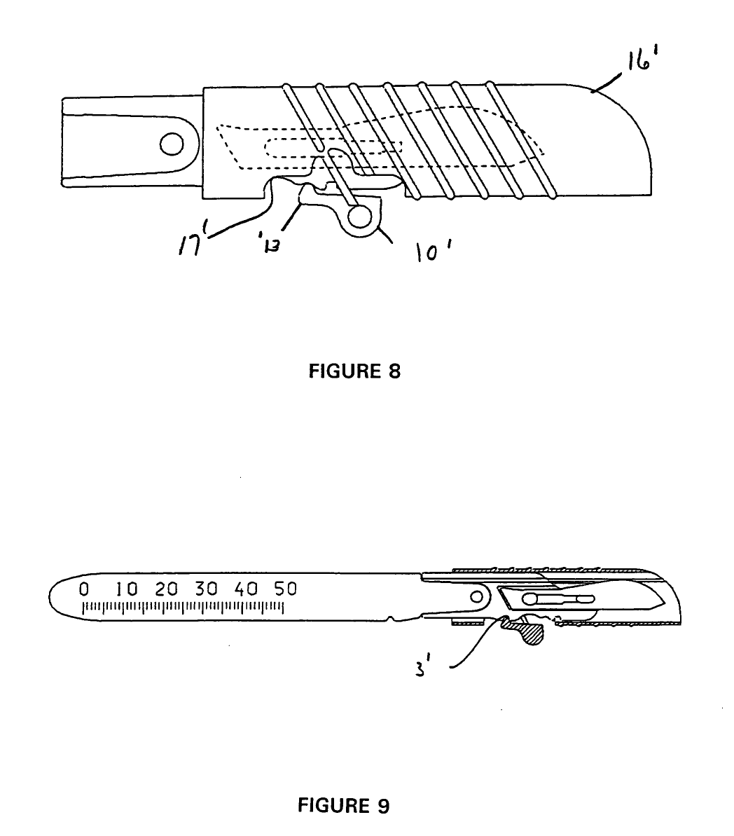

FIGS. 8 and 9 illustrate a second embodiment of the pre-assembly cartridge 22′ and its use in a completed scalpel. According to this embodiment sleeve 16′ has a retention tooth 13′ that is pivotally supported by the sleeve 16′ below the opening 17′ on the underside of the sleeve. It is a further aspect of this embodiment that the notch 3′ on the blade carrier 12′ is modified, but functions according to the same sequence as previously described with respect to the embodiment of FIG. 1.

The embodiments shown in FIGS. 8 and 9 address the risk posed when a scalpel having a heavy stainless steel handle is dropped or falls onto the floor or other hard surface. When a scalpel strikes such a surface the impact might cause the blade to be accidentally exposed. Thus, it is an aspect of the second embodiment of retention tooth 13′ and notch 3′ as illustrated in FIGS. 8 and 9 to provide a more robust connection and substantially reduce the risk of accidental exposure.

According to this embodiment, the sleeve 16′ is moved along the blade carrier 14′ and handle 24′ by depressing the button 10′. This tooth and button configuration 10′, 13′ is somewhat different than the previously described configuration, in that the legs 15′ are more rigid, and the movement of the tooth 13 is pivotal about the axis of the button 10, rather than from bending of the legs 15. As shown in FIG. 9, when the retention tooth 13′ is engaged with the notch 3′, the steep angle of the notch 3′ prevents accidental blade exposure in the event the instrument is dropped. With this alternative configuration, the blade carrier 14′ remains attached to the handle 24′ with the blade 12′ still protected. In order to move the sleeve 16′ to expose the blade 12′ the button 10′ adjacent to the retention tooth 13′ must be depressed.

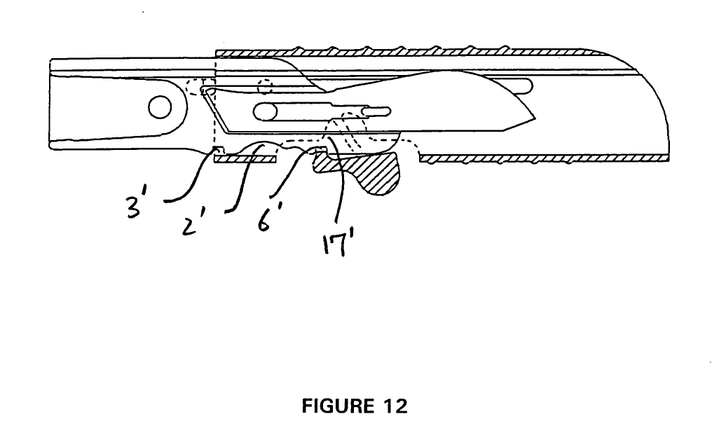

In the embodiment of FIG. 12, the opening 17′ is formed in the lower distal portion of the sleeve, leaving a hood or similar overhang at the upper distal portion of the sleeve, for covering the mounting formation at the proximal portion of the handle. This embodiment of the sleeve is easier to manufacture.

FIGS. 14A-14E illustrate the preferred method for assembling the safety scalpel as exemplified by the embodiments of FIGS. 8, 9 and 12. According to this aspect of the invention, numerous sleeves 16 are placed on an assembly rod 32. The assembly rod 32 preferably has means, such as a protrusion on the bottom, for preventing the sleeves from slipping off the assembly rod. The protrusion 30 temporarily catches the retention tooth 13 of the lead sleeve. It is also preferred that the leading end of the assembly rod 32 is shaped like the mounting end 25 of handle 24 and that the top edge of the assembly rod have a guide channel 18′ that is complementary to the guide track 19 of the blade carrier 14. The assembly rod 32 has a mounting tongue 7′ and a locating bore 26′ to engage the proximal end of one of a series of blade carriers 14 that have been pre-assembled with respective blades 12. Together, the blade carrier 14 and blade 12 form an elongated blade assembly 33 that is temporarily attached to the assembly rod 32. Once the elongated blade assembly 33 is attached, the sleeve 16 can be moved to its final position over the blade 12. In its final position, retention tooth 13 is resting in notch 2 of the blade carrier 14. The completed pre-assembly cartridge 22 is then removed from the assembly rod 32 and is ready for sterilization and packaging. The next sleeve is then ready for attachment to the next of the ready supply of blade assemblies 33.

1. A surgical scalpel comprising;
a blade carrier having a distal portion, a proximal portion, and a lower edge defining a plurality of notches;
a blade rigidly affixed to and extending from the distal portion of the carrier;
a handle having a distal portion removably attached to the proximal portion of the carrier and a lower edge having a notch; and
a substantially tubular sleeve surrounding the blade carrier and the blade, having a proximal end, a distal end, and an underside that has a depending tooth for engaging the notches on the carrier and the handle whereby the user can slide the sleeve and position the tooth for selectively establishing and reestablishing at least three detent conditions
(1) first condition wherein the tooth engages at least one of the notches on the blade carrier, the sleeve distal end covers the blade, and the sleeve proximal end covers the distal portion of the blade carrier but not the proximal portion of the blade carrier where the handle is attachable to the blade carrier,
(2) a second condition wherein the tooth engages another notch on the blade carrier, the sleeve distal end covers the blade, and the sleeve proximal end extends over the front portion of the handle to secure the attachment of the handle to the blade carrier, and
(3) a third condition wherein the tooth engages the notch on the handle, the sleeve spans the handle and the blade carrier, and the distal end of the sleeve is behind the blade to expose the blade for cutting.

2. The surgical scalpel of claim 1, wherein the blade carrier includes a locking notch and the user can slide the sleeve from any of the conditions (1), (2) or (3) to establish a locked condition (4) wherein the tooth engages the locking notch and the sleeve cannot thereafter slide back to any of the conditions (1), (2) or (3).

3. The surgical scalpel of claim 1, wherein the underside of the sleeve has an opening and the tooth depends from the sleeve beneath the opening.

4. The surgical scalpel of claim 3, wherein the opening is intermediate the ends of the sleeve.

5. The surgical scalpel of claim 3, wherein the opening is at the proximal end of the sleeve.

6. The surgical scalpel of claim 1, wherein the blade carrier has opposed lateral sides and the blade and the handle are attached to the same lateral side of the blade carrier, offset from the blade carrier centerline.

7. The surgical scalpel of claim 1, wherein the blade carrier has opposed lateral sides and at least one guide channel along at least one of said lateral sides.

8. The surgical scalpel of claim 6, wherein
the proximal end of the carrier has an elongated mounting pocket and a lateral projection in said pocket,
the distal end of the handle has an elongated tongue with a hole that nests on said pocket with the projection in said hole; and
a distal portion of said sleeve covers the pocket when the sleeve is in said second and third conditions.

9. The surgical scalpel of claim 8, wherein the blade carrier has three notches including a locking notch closest to the distal end of the carrier, and the user can slide the sleeve from any of the conditions (1), (2) or (3) to establish a locked condition (4) wherein the tooth engages the locking notch and the sleeve cannot thereafter slide back to any of the conditions (1), (2) or (3).

10. The surgical scalpel of claim 9, wherein
the underside of the sleeve has an opening and the tooth depends from the sleeve beneath the opening; and
the tooth is biased into the lower edge of the carrier; and
a button is integrally associated with the tooth for selective manual pivoting of the tooth away from said bottom edge whereby the sleeve can be slid to any of said first, second, and third conditions.

11. A sub-assembly cartridge for a surgical scalpel comprising:
a substantially flat blade carrier having a front portion, a rear portion, opposed sides, and a lower edge, one of said sides having a first, distal recess with first lateral projection and said same side having a second, proximal recess with a second lateral projection, wherein said edge defines a plurality of notches at least including a proximal notch, a distal notch, and an intermediate notch and having at least one guide channel opposite said lower edge;
a blade extending from the front portion of the blade carrier, seated in said first recess and rigidly affixed to said first projection, which together with the blade carrier defines an elongated blade assembly; and
a substantially tubular protective sleeve having a proximal end and a distal end and an underside that has a resilient tooth engaging the intermediate notch, wherein the distal end of the sleeve projects beyond the blade and the proximal end including said second proximal recess of the blade carrier projects from the proximal end of the sleeve, said sleeve being slidable longitudinally along the blade assembly to reposition said tooth in either of said proximal or distal notches.

12. The sub-assembly cartridge of claim 11, where when said tooth is repositioned in the proximal notch, the proximal end of the sleeve overlaps the second projection of the second recess in the carrier.

13. The sub-assembly cartridge of claim 11, wherein when said tooth is repositioned in the distal notch, the distal end of the sleeve extends beyond the blade and tooth cannot be repositioned to either of the intermediate or proximate notches.

14. The sub-assembly cartridge of claim 11, wherein when said tooth is repositioned in the distal notch, the distal end of the sleeve extends beyond the blade and tooth cannot be repositioned to either of the intermediate or proximate notches.

15. The sub-assembly of claim 11, wherein the underside of the sleeve has an opening and the tooth depends from the sleeve beneath the opening.

16. The sub-assembly of claim 15, wherein
the underside of the sleeve has an opening and the tooth depends from the sleeve beneath the opening;
the tooth is biased into the lower edge of the carrier; and
a button is operatively associated with the tooth for selective manual pivoting of the tooth away from said bottom edge whereby the sleeve can be slid to any of said first, second, and third conditions.

17. A method for assembling a surgical scalpel comprising:
providing an assembly rod with a top edge and a distal end having a first, distal recess with a first lateral projection;
sliding a plurality of sleeves each having a tooth onto said assembly rod;
attaching the proximal end of an elongated blade assembly having a plurality of notches along one edge to said distal end of said assembly rod;
sliding said one sleeve over said proximal end of said elongated blade assembly such that said tooth engages one of said notches;
removing said elongated blade assembly together with said one sleeve.

18. The method of claim 17 wherein said assembly rod has a retaining means adjacent to said distal end.

19. The method of claim 18 wherein the tooth of at least one of said plurality of sleeves temporarily engages said retaining means.

20. The method of claim 17 wherein said top edge of said assembly rod has at least one guide channel.
